SEQUENCE LISTING (90 sequences)

SEQ ID NO: 1 - DNA, 53 bp, Canis lupus
  source 1..53: mol_type = genomic DNA
gctccgggttcggggggggcgggaggcgaaagggatccgcgctcccagacccc

SEQ ID NO: 2 - DNA, 53 bp, Canis lupus
  source 1..53: mol_type = genomic DNA
ccacctgtccgggcctttccggggtgcgtgggcggcgcctcggcgggcggagc

SEQ ID NO: 3 - DNA, 53 bp, Canis lupus
  source 1..53: mol_type = genomic DNA
ccgcgccaggcgcagcacggcctccacggcgatggtgaagcagagcgcgctga

SEQ ID NO: 4 - DNA, 53 bp, Canis lupus
  source 1..53: mol_type = genomic DNA
ccccgggggcggtggtgcttcctctgcgcgccccgccagtcggtgcccgggct

SEQ ID NO: 5 - DNA, 53 bp, Canis lupus
  source 1..53: mol_type = genomic DNA
gttactcaggcgtagcttgtgccagacgacttctggccagcgttttcaagggg

SEQ ID NO: 6 - DNA, 53 bp, Canis lupus
  source 1..53: mol_type = genomic DNA
gcggcggtgccgagagtcgaggggcgcggagtctgcgagcggggaagttcagc

SEQ ID NO: 7 - DNA, 53 bp, Canis lupus
  source 1..53: mol_type = genomic DNA
gtggctggagccgcgcgagccagaaccgcgggggcaggtcctcgcagggcccg

SEQ ID NO: 8 - DNA, 53 bp, Canis lupus
  source 1..53: mol_type = genomic DNA
gggcgccttcttctcccggggccttccgcctgctatcagctgttaaggctggg

SEQ ID NO: 9 - DNA, 53 bp, Canis lupus
  source 1..53: mol_type = genomic DNA
cctccgcgtggtctctgcccgccttgcgccccctctcagcgtcctctctctct

SEQ ID NO: 10 - DNA, 53 bp, Canis lupus
  source 1..53: mol_type = genomic DNA
cagttatctgaaaagtttctggccgccgggagaggatgaatttcaaggtggca

SEQ ID NO: 11 - DNA, 53 bp, Canis lupus
  source 1..53: mol_type = genomic DNA
gggacgcctcccagagtttagacggacgaacccaggctcgctaggacgcccag

SEQ ID NO: 12 - DNA, 53 bp, Canis lupus
  source 1..53: mol_type = genomic DNA
ccaacgtgcttgccaaggcgctgagccgcggcgttctgcaccaagacaaggat

SEQ ID NO: 13 - DNA, 53 bp, Canis lupus
  source 1..53: mol_type = genomic DNA
tggaggggccgaaggggtgctccatccgcggcccgcggcggggaagggggtcc

SEQ ID NO: 14 - DNA, 53 bp, Canis lupus
  source 1..53: mol_type = genomic DNA
ggcgccgggtgctgccggaggggcggcggggggcgcccgggccgctggcgagg

SEQ ID NO: 15 - DNA, 53 bp, Canis lupus
  source 1..53: mol_type = genomic DNA
actcctactatctaccaggcctaacccgtttgacatatcacaggccccctgcc

SEQ ID NO: 16 - DNA, 53 bp, Canis lupus
  source 1..53: mol_type = genomic DNA
ggcgtccgcggggagccggggctcttcgccggccggacccacacccgccggct

SEQ ID NO: 17 - DNA, 53 bp, Canis lupus
  source 1..53: mol_type = genomic DNA
ccgcccagcgccgcccggagcagccgcggccccgcgaggagacgggcgccgcc

SEQ ID NO: 18 - DNA, 53 bp, Canis lupus
  source 1..53: mol_type = genomic DNA
ccttccggagcaagggagccccgagtcgcgggtcccacgcccgccacccgagg

SEQ ID NO: 19 - DNA, 53 bp, Canis lupus
  source 1..53: mol_type = genomic DNA
gacagtacccacgaaaagggggcagacgtttggcctgaggggcagcaggcacg

SEQ ID NO: 20 - DNA, 53 bp, Canis lupus
  source 1..53: mol_type = genomic DNA
gcccgcggccccgcgcggccagccctcgctcggctcagcgtcgatgcccctgt

SEQ ID NO: 21 - DNA, 53 bp, Canis lupus
  source 1..53: mol_type = genomic DNA
tccccgccgcgtgcacggtgcgctcccgcccagcgcccgaccccgcccgccgc

SEQ ID NO: 22 - DNA, 53 bp, Canis lupus
  source 1..53: mol_type = genomic DNA
ctaggatccagactgcccgcagacaccgctgcccagtccttggccccttcctt

SEQ ID NO: 23 - DNA, 53 bp, Canis lupus
  source 1..53: mol_type = genomic DNA
ggcaggcggggggcggcacacatgcccggattgtgacgtccagtctgttgctg

SEQ ID NO: 24 - DNA, 53 bp, Canis lupus
  source 1..53: mol_type = genomic DNA
ggcgggggcgagaggcggtcccggctcgggcactgcggctccgccgggagccc

SEQ ID NO: 25 - DNA, 53 bp, Canis lupus
  source 1..53: mol_type = genomic DNA
cccgaccgggctggtgaccctgaggccgggtgcctggcggtctgtaccctgca

SEQ ID NO: 26 - DNA, 53 bp, Canis lupus
  source 1..53: mol_type = genomic DNA
aaccggtgagaaagcaggtggaacagcgggaggtttccccttctctgaatgaa

SEQ ID NO: 27 - DNA, 53 bp, Canis lupus
  source 1..53: mol_type = genomic DNA
tgaacagcaggacgagaactttgtggcgtagcgtgaacgcgcttcaggatcag

SEQ ID NO: 28 - DNA, 53 bp, Canis lupus
  source 1..53: mol_type = genomic DNA
tgtgatttacttccctgctttatccccgacttgagccactgggcttaaaaaag

SEQ ID NO: 29 - DNA, 53 bp, Canis lupus
  source 1..53: mol_type = genomic DNA
cccgcagccccgtgcgcggagccgcgcggcccccggctgcagctccgggctgg

SEQ ID NO: 30 - DNA, 53 bp, Canis lupus
  source 1..53: mol_type = genomic DNA
acctactgtgtgcaagcacccagctccgtgcgatagaggagccccggcctcta

SEQ ID NO: 31 - DNA, 53 bp, Canis lupus
  source 1..53: mol_type = genomic DNA
tgtccctttcatacggaagggaaaaccgaggcccaagagagcgtaggtgggac

SEQ ID NO: 32 - DNA, 53 bp, Canis lupus
  source 1..53: mol_type = genomic DNA
ttttcgtgcgagggggcctccagccgcgctcagcccgcccctcgtctcctctt

SEQ ID NO: 33 - DNA, 53 bp, Canis lupus
  source 1..53: mol_type = genomic DNA
gaggcagtccaggccttgttaggtagcgctgggaatcacaagtggggtggctt

SEQ ID NO: 34 - DNA, 53 bp, Canis lupus
  source 1..53: mol_type = genomic DNA
ccacgcgtcttcacgcggcaggcaggcgttccaggcccatttcacagatgaag

SEQ ID NO: 35 - DNA, 53 bp, Canis lupus
  source 1..53: mol_type = genomic DNA
agtcagagggctgcttgagggactctcgctggtaagaagtcgatctgaaacag

SEQ ID NO: 36 - DNA, 53 bp, Canis lupus
  source 1..53: mol_type = genomic DNA
ctcaagagtaagtttctgatggagctcggaaccctgagggtcaactttaccca

SEQ ID NO: 37 - DNA, 53 bp, Canis lupus
  source 1..53: mol_type = genomic DNA
cagccccgccgccggctggggccctccgggagcccggggcgaccccagccccc

SEQ ID NO: 38 - DNA, 53 bp, Canis lupus
  source 1..53: mol_type = genomic DNA
ccgcagcgcagtccgcgggcaggaggcgtcacccgaaggaacagccgcgagga

SEQ ID NO: 39 - DNA, 53 bp, Canis lupus
  source 1..53: mol_type = genomic DNA
tttcttacttctatgctagggtttcccggttgcacctacattttaaaagtgtt

SEQ ID NO: 40 - DNA, 53 bp, Canis lupus
  source 1..53: mol_type = genomic DNA
tgtgccacagaggggtccgcaggcgtcggacagacacgggccgaggagaattc

SEQ ID NO: 41 - DNA, 53 bp, Canis lupus
  source 1..53: mol_type = genomic DNA
tgtttgagctgaagaaacgagccaaacgcctcgctgggatgcccacagggagt

SEQ ID NO: 42 - DNA, 53 bp, Canis lupus
  source 1..53: mol_type = genomic DNA
tctggttccccttggactcccacgctcgagcccgtaagtaggcagaggaggga

SEQ ID NO: 43 - DNA, 53 bp, Canis lupus
  source 1..53: mol_type = genomic DNA
gaggggatgggaccgaggcacgtggacgtgggagcaatagtgacccaagggat

SEQ ID NO: 44 - DNA, 53 bp, Canis lupus
  source 1..53: mol_type = genomic DNA
gcccttgccaattcaggaattctcggcggggtctgcacacacagtttcacgca

SEQ ID NO: 45 - DNA, 53 bp, Canis lupus
  source 1..53: mol_type = genomic DNA
gacagatccgctgccctcacagtaaccgggctgccagagctgctgacccggcc

SEQ ID NO: 46 - DNA, 53 bp, Canis lupus
  source 1..53: mol_type = genomic DNA
ccataactgatgcccacagggtctgacgttctgagttaacctgcattataact

SEQ ID NO: 47 - DNA, 53 bp, Canis lupus
  source 1..53: mol_type = genomic DNA
ccgttcttctgctgcggagggtcgcacgaggcctcgaacacggcgaggcgcgg

SEQ ID NO: 48 - DNA, 53 bp, Canis lupus
  source 1..53: mol_type = genomic DNA
gtcggcccggggccgggcaagctcggcggccagcgcggaggcggcggcgggag

SEQ ID NO: 49 - DNA, 53 bp, Canis lupus
  source 1..53: mol_type = genomic DNA
gagcggccgccagtggccgcggccggcgcgcggtggaggccggcgccgctgct

SEQ ID NO: 50 - DNA, 53 bp, Canis lupus
  source 1..53: mol_type = genomic DNA
cgctgctggctcagccctaggtcttccgcctctgtaaatgtttgttggaggga

SEQ ID NO: 51 - DNA, 53 bp, Canis lupus
  source 1..53: mol_type = genomic DNA
tacaaacacccagaaacgctggggggcgggggaggggcacgggtgcgcccctc

SEQ ID NO: 52 - DNA, 53 bp, Canis lupus
  source 1..53: mol_type = genomic DNA
gccccgcatccagggcagggggactgcgactgccagaggctgagcaactcgcc

SEQ ID NO: 53 - DNA, 53 bp, Canis lupus
  source 1..53: mol_type = genomic DNA
ccagtctgaaacggctgctaggaacccgatcaaacccttcacaaattttctca

SEQ ID NO: 54 - DNA, 53 bp, Canis lupus
  source 1..53: mol_type = genomic DNA
accctcgggggccctaccggcagcgccgccggagcttgagccgcgccgaccgt

SEQ ID NO: 55 - DNA, 53 bp, Canis lupus
  source 1..53: mol_type = genomic DNA
gctccagcgccccgggcctgtctgggcggccgggagcgccccgcctgggacca

SEQ ID NO: 56 - DNA, 53 bp, Canis lupus
  source 1..53: mol_type = genomic DNA
cgggctcggcacttggcagaggttcgcgttcccaggtgccctggggcccccgc

SEQ ID NO: 57 - DNA, 53 bp, Canis lupus
  source 1..53: mol_type = genomic DNA
gctgaagcctatggtcttcctgcctgcgacacgcgtttcattggcaagcaagt

SEQ ID NO: 58 - DNA, 53 bp, Canis lupus
  source 1..53: mol_type = genomic DNA
ccggcccccgccctcacactgactttcggaagatacaggggaagaaggggtcc

SEQ ID NO: 59 - DNA, 53 bp, Canis lupus
  source 1..53: mol_type = genomic DNA
cctcggggctcaggccaagcccacagcgcgcgggggagggatgctcgggcagg

SEQ ID NO: 60 - DNA, 53 bp, Canis lupus
  source 1..53: mol_type = genomic DNA
gcaggggaattggacccgcggggactcgcgccttcccggaccggcggcgggga

SEQ ID NO: 61 - DNA, 53 bp, Canis lupus
  source 1..53: mol_type = genomic DNA
gtgcctaggtaacagtccacctaacgcgaggtgcaggccagctctcccaagat

SEQ ID NO: 62 - DNA, 53 bp, Canis lupus
  source 1..53: mol_type = genomic DNA
aagctcccccgtctcccgctgacgggcggccggctgcaggcgtcgggacgcca

SEQ ID NO: 63 - DNA, 53 bp, Canis lupus
  source 1..53: mol_type = genomic DNA
aaattgggacttgaacccagttctgacgctggagaaagcgattttaacactat

SEQ ID NO: 64 - DNA, 53 bp, Canis lupus
  source 1..53: mol_type = genomic DNA
cgtgtgcagccgcgcccagcacgggccgcccctcccggacctgagctcgcgcc

SEQ ID NO: 65 - DNA, 53 bp, Canis lupus
  source 1..53: mol_type = genomic DNA
gggcgccgcgcccgcggggagctcgccgcccgggccgccctcgggcccccgcg

SEQ ID NO: 66 - DNA, 53 bp, Canis lupus
  source 1..53: mol_type = genomic DNA
gctggtccagggtgggctgcagggcccgggacacttgcttggccctctccccg

SEQ ID NO: 67 - DNA, 53 bp, Canis lupus
  source 1..53: mol_type = genomic DNA
ggcacgcacccccggaggcgaggtgtcggccagaggggggaccgtcagggggt

SEQ ID NO: 68 - DNA, 53 bp, Canis lupus
  source 1..53: mol_type = genomic DNA
cgcccccttcccagcctcagcggcctcgcctgggcctccgccagcgtgagcac

SEQ ID NO: 69 - DNA, 53 bp, Canis lupus
  source 1..53: mol_type = genomic DNA
gggccgcaggactgctcgaaaggcatcggattacccagaataactcccaaata

SEQ ID NO: 70 - DNA, 53 bp, Canis lupus
  source 1..53: mol_type = genomic DNA
cggcagggccggagcgggggactggccggctggacccagggcgccgacccacc

SEQ ID NO: 71 - DNA, 53 bp, Canis lupus
  source 1..53: mol_type = genomic DNA
acctgctgcgcatgaaggtgcggggacggcggggaggcgcgctgggcgacggg

SEQ ID NO: 72 - DNA, 53 bp, Canis lupus
  source 1..53: mol_type = genomic DNA
ggccgtggggctcaggtgtgagcggccgccgaggcgcgcgccccgagtgtgcg

SEQ ID NO: 73 - DNA, 53 bp, Canis lupus
  source 1..53: mol_type = genomic DNA
gccagtgttcccaagagatgctcagccgctagtgacaggtttagctccacttt

SEQ ID NO: 74 - DNA, 53 bp, Canis lupus
  source 1..53: mol_type = genomic DNA
ggccctgctgcccggccctggccgcccggcctcagatcttcccccggcgggcg

SEQ ID NO: 75 - DNA, 53 bp, Canis lupus
  source 1..53: mol_type = genomic DNA
tccgctccctctcccgacccccgccgcggtcttccgcttactcacaggggtag

SEQ ID NO: 76 - DNA, 53 bp, Canis lupus
  source 1..53: mol_type = genomic DNA
gccgcgctggcgggaccccgtgactgcggaccgcgggccctctccgatgcact

SEQ ID NO: 77 - DNA, 53 bp, Canis lupus
  source 1..53: mol_type = genomic DNA
gcttgaccaggggccgctgagcaggacgtgctctgcaggcacactaagctcac

SEQ ID NO: 78 - DNA, 53 bp, Canis lupus
  source 1..53: mol_type = genomic DNA
agagacagtgatgaggaatgagctgccgaaggaatttggcaaggaggggagcg

SEQ ID NO: 79 - DNA, 53 bp, Canis lupus
  source 1..53: mol_type = genomic DNA
tatctctgcgtgctttgcatcctagtcgaattgcttagttggatgaaatgtca

SEQ ID NO: 80 - DNA, 53 bp, Canis lupus
  source 1..53: mol_type = genomic DNA
ccgcgctcgggcgaggggccaggccgcgctggcgggaccccgtgactgcggac

SEQ ID NO: 81 - DNA, 53 bp, Canis lupus
  source 1..53: mol_type = genomic DNA
gcgctccgtgctgggtgctccgcgctcggtgctccgcgcccccactcggtgct

SEQ ID NO: 82 - DNA, 53 bp, Canis lupus
  source 1..53: mol_type = genomic DNA
cagctccctcgagagagtgttctctgcggtgaccaggaaggtgggaaggtgct

SEQ ID NO: 83 - DNA, 53 bp, Canis lupus
  source 1..53: mol_type = genomic DNA
caacggggtcgcagcagcgcacagtgcgcgggaaccctgctttcccgaggcag

SEQ ID NO: 84 - DNA, 53 bp, Canis lupus
  source 1..53: mol_type = genomic DNA
ggagagcgactgcgcacgcgccgggtcgagttctgggcaccacgtttagctcc

SEQ ID NO: 85 - DNA, 53 bp, Canis lupus
  source 1..53: mol_type = genomic DNA
tcgggcatctcaggcatccgagggggcggcctggctgtgcgcagaggagccca

SEQ ID NO: 86 - DNA, 53 bp, Canis lupus
  source 1..53: mol_type = genomic DNA
gtgggagcaggaggtcagcgcaggcccgtcgcccccggggaaaagcggggtac

SEQ ID NO: 87 - DNA, 53 bp, Canis lupus
  source 1..53: mol_type = genomic DNA
ggccgagccccctccccacgctgccccgctggacacgagggcgcaggcgggag

SEQ ID NO: 88 - DNA, 53 bp, Canis lupus
  source 1..53: mol_type = genomic DNA
agaattcttctatgactcgtctttttcgttcccttcgtttatttcccgaagtc

SEQ ID NO: 89 - DNA, 53 bp, Canis lupus
  source 1..53: mol_type = genomic DNA
gtcagctcttccggacgtggcgagttcgcctctgcccgaaactcatcccgcgt

SEQ ID NO: 90 - DNA, 53 bp, Canis lupus
  source 1..53: mol_type = genomic DNA
gcatggagcacatcacctaagtaagccggccttccgttggcggacagcccgag

(19) United States
(12) Patent Application Publication
(10) Pub. No.: US 2026/0103756 A1
(43) Pub. Date: Apr. 16, 2026

(54) METHODS RELATED TO OSTEOARTHRITIS USING DNA METHYLATION PROFILES OF CANINES

(71) Applicant: SOCIETE DES PRODUITS NESTLE S.A., VEVEY (CH)

(72) Inventors: Alix ZOLLINGER, Denges (CH); Lorane TEXARI, Morges (CH); Rondo MIDDLETON, Creve Coeur, MO (US); Eleonora PORCU, Lausanne (CH)

(21) Appl. No.: 19/324,741

(22) Filed: Sep. 10, 2025

Related U.S. Application Data
(60) Provisional application No. 63/705,828, filed on Oct. 10, 2024.

(51) Int. Cl. C12Q 1/6883

(52) U.S. Cl. CPC C12Q 1/6883; C12Q 2600/118; C12Q 2600/154

(57) ABSTRACT

The present invention provides a method of determining whether a canine subject has or is at a risk of developing osteoarthritis (OA), the method comprising:
a) providing a DNA methylation profile from a sample obtained from the canine subject; and
b) determining whether the canine subject has or is at risk of developing OA by comparing at least part of the DNA methylation profile to a reference DNA methylation profile.

### CROSS REFERENCE TO RELATED APPLICATIONS

This application claims the benefit of and priority to U.S. Provisional Application Ser. No. 63/705,828 filed Oct. 10, 2024, the disclosure of which is incorporated in its entirety herein by this reference.

### SEQUENCE LISTING

The instant application contains a Sequence Listing which has been submitted electronically in XML file format and is hereby incorporated by reference in its entirety. Said XML copy, created on Sep. 3, 2025, is named 19471-US-NP_SL and is 82,406 bytes in size.

### FIELD OF THE INVENTION

The present invention relates to a method for determining if a canine subject has, or is at a risk of developing, osteoarthritis (OA) using a DNA methylation profile. The invention further relates to methods of selecting a dietary, lifestyle or pharmacological regime for a canine subject, for example to prevent or reduce the risk of the canine subject developing OA, based on an OA risk determined from a DNA methylation profile according to the present invention.

### BACKGROUND

Musculoskeletal pain and lameness are among the most common clinical problems seen in companion dogs. A large proportion of these cases involve osteoarthritis (OA), the most common joint disorder in dogs.

OA is a progressive, incurable condition that can affect comfort and quality of life for a substantial proportion of a patient's life. This condition compromises the welfare of affected dogs and places a significant burden on human caregivers. Musculoskeletal pain and locomotor dysfunction, often due to OA, are also among the most frequent reasons for euthanasia in dogs.

Currently, the primary approach to mitigating the negative impact of canine OA is treatment once clinical signs have manifested. Earlier detection and interventions to prevent or delay the development of OA could have significant benefits. Effective approaches for preventing or delaying the development of OA depend on a clear understanding of relevant risk factors and identifying individuals at increased risk.

Age, higher adult body weight, gonadectomy, and younger age at gonadectomy have been found to be significantly associated with higher risks of OA (Graves et al.; Front. Vet. Sci.; 10; 2023). There is also evidence for the role of genetic factors influencing both individual risk and breed differences in susceptibility to OA. However, these factors are often associated indirectly with OA causing predisposition conditions such as hip or elbow dysplasia or a propensity for cranial cruciate ligament rupture, which then leads to the development of arthritis.

Accordingly, there is a need for further methods for determining the risk or likelihood of a dog developing OA.

### SUMMARY

In a first aspect, the present invention provides a method of determining if a canine subject has, or is at a risk of developing osteoarthritis (OA), the method comprising:
a) providing a DNA methylation profile from a sample obtained from the canine subject; and
b) determining whether the canine subject has or is at risk of developing OA by comparing at least part of the DNA methylation profile to a reference DNA methylation profile.

The present invention provides markers that allow it to be determined whether a dog has, or is at risk of developing, OA. Advantageously, a dog that is determined to have, or be at risk of developing, OA may be provided with a dietary, pharmacological, or lifestyle regime aimed at treating, or reducing the risk of the dog developing, OA.

Suitably, the present methods may be used to determine whether a dog is at risk of developing OA. Suitably, the present methods may be used to determine a risk that a dog will develop OA. In particular, the present methods may enable a dog at risk of developing OA to be identified at an earlier stage than traditional methods, and thus a dietary, pharmacological and/or lifestyle regime to be applied at an earlier stage.

Accordingly, in further aspects the present invention provides a method for selecting a dietary, pharmacological, or lifestyle regime for a canine subject, the method comprising:
a) providing a DNA methylation profile from a sample obtained from the canine subject;
b) determining whether the canine subject has or is at risk of developing osteoarthritis (OA) by comparing at least part of the DNA methylation profile to a reference DNA methylation profile; and
c) selecting a suitable dietary, pharmacological, or lifestyle regime for the canine subject based on the whether the canine subject has, or is at risk of developing OA, as determined in step b).

As used herein, ‘selecting a suitable dietary, pharmacological or lifestyle regime for a dog’ may also encompass ‘recommending a dietary, pharmacological or lifestyle regime for the dog’ or ‘providing a recommended dietary, pharmacological or lifestyle regime for the dog’.

The present invention further provides a method for preventing or reducing the risk of a canine subject developing osteoarthritis (OA); the method comprising:
a) providing a DNA methylation profile from a sample obtained from the canine subject;
b) determining a risk of the canine subject developing osteoarthritis (OA) by comparing at least part of the DNA methylation profile to a reference DNA methylation profile; and
c) selecting a dietary, pharmacological, or lifestyle regime for the canine subject based on the risk determined in step b);
wherein the dietary, pharmacological, or lifestyle regime prevents or reduces the risk of the canine subject developing OA.

The method may further comprise administering the selected dietary, pharmacological, or lifestyle regime to the canine subject.

The DNA methylation profile may be any DNA methylation profile as described herein.

The present invention further provides a dietary or pharmaceutical product for use in reducing the risk that a canine subject will develop OA, wherein the dietary or pharmaceutical product is administered to a canine subject that has been determined to be at risk of developing OA according to the method of the present invention.

The present invention further provides a computer-readable medium comprising instructions that when executed cause one or more processors to perform the method of the invention.

The invention also provides a computer system for determining whether a canine subject has or is at risk of developing osteoarthritis (OA), the computer system programmed to compare at least part of a DNA methylation profile obtained from the canine subject to a reference DNA methylation profile; and optionally (ii) determine if the canine subject has, or is at risk of developing, OA by comparing at least part of the DNA methylation profile to a reference DNA methylation profile.

The invention further provides a computer system for selecting a dietary, pharmacological, or lifestyle regime for a canine subject, the computer system programmed to perform the steps of: a) determining whether a canine subject has or is at risk of developing osteoarthritis (OA) by comparing at least part of a DNA methylation profile obtained from the canine subject to a reference DNA methylation profile; and b) selecting a suitable dietary, pharmacological, or lifestyle regime for the canine subject based on whether the canine subject has, or is at risk of developing OA, as determined in step a).

The invention also provides a computer program product comprising computer implementable instructions for causing a programmable computer to determine whether a canine subject has or is at risk of developing osteoarthritis (OA), by comparing at least part of a DNA methylation profile obtained from the canine subject to a reference DNA methylation profile; and (ii) determining whether the canine subject has, or is at risk of developing OA, using the comparison of the at least part of the DNA methylation profile to a reference DNA methylation profile.

The invention further provides a computer program product comprising computer implementable instructions for causing a programmable computer to select a dietary, pharmacological, or lifestyle regime computer program product comprising computer implementable instructions for causing a programmable computer to select a dietary, pharmacological, or lifestyle regime for a canine subject by a) whether the canine subject has, or is at risk of developing osteoarthritis (OA) by comparing at least part of a DNA methylation profile obtained from the canine subject to a reference DNA methylation profile; and b) selecting a suitable dietary, pharmacological, or lifestyle regime for the canine subject based on whether the canine subject has, or is at risk of developing OA, as determined in step a).

### DESCRIPTION OF THE DRAWINGS

FIG. 1—Predicted probability of developing OA (OA risk) for dogs on a testing set using an illustrative methylation-based model which uses 90 methylation sites. The model predicts a significantly higher risk for dogs that indeed developed OA (OA TRUE) compared to dogs that did not develop OA (OA FALSE).

FIG. 2—Predicted probability of developing OA (OA risk score) for dogs on a validation set using an illustrative methylation-based model which uses 90 methylation sites. The model predicts a significantly higher risk for dogs that developed OA (TRUE) compared to dogs that did not develop OA (FALSE).

### DETAILED DESCRIPTION

### Subject

The present methods are directed to canine subjects. Accordingly, the subject of the present invention is a dog, suitably a domestic dog.

The subject may have or be at risk of OA, as described herein. ‘At risk of OA’ refers to a subject who is not showing any symptoms of OA. The subject may have a predisposition for, or be thought to be at risk of developing, OA.

Suitably, a subject at risk of OA may have a higher propensity, likelihood and/or probability, to develop OA than average. For example, a subject at risk or OA may have a higher propensity, likelihood and/or probability, to develop OA than a corresponding dog of the same age and/or breed.

Suitably, a subject at risk of OA may have a higher probability to develop OA than average. For example, a subject at risk or OA may have a higher probability to develop OA than a corresponding dog of the same age and/or breed.

### Sex

Suitably, the sex of the dog may be classified as male or female. Suitably, the sex of the dog may be included in the present methods (e.g. in a model as described herein).

### Chronological Age

Chronological age may be defined as the amount of time that has passed from the subject's birth to the given date. Chronological age may be expressed in terms of years, months, days, etc.

Suitably, the present method may be applied to a dog of any chronological age.

Suitably, the chronological age of the dog may be included in the present methods (e.g. in a model as described herein).

### Biological Age

Depending on genetics, nutrition, and lifestyles—for example—individuals may age slower or faster than their chronological age. Chronological age may therefore not always reflect an individual's biological rate of aging. As such, the biological age of an individual (based on e.g. clinical biochemistry and cell biology measures) can vary compared to others of the same chronological age. Methods for determining biological age are known in the art and include, for example, methods utilising methylation profiles, clinical chemistry panel profiles or telomere length.

Suitably, the present method may be applied to a dog of any biological age. Suitably, the biological age of the dog may be included in the present methods (e.g. in a model as described herein).

### Sample

The present invention may comprise a step of providing a DNA methylation profile from one or more samples obtained from a canine subject.

The present invention may comprise a step of determining a DNA methylation profile from one or more samples obtained from a canine subject.

Suitably, the sample may be a blood, hair follicle, buccal swab, saliva, fecal or tissue sample.

Suitably, the sample is derived from blood. The sample may contain a blood fraction or may be whole blood. The sample preferably comprises whole blood. The sample may comprise a peripheral blood mononuclear cell (PBMC) or lymphocyte sample. Techniques for collecting samples from a subject and extracting DNA (e.g. genomic DNA) from the sample are well known in the art.

Suitably, the sample is a hair follicle, buccal swab or saliva sample. Such sample types are particularly applicable if the sample is to be provided, for example, outside of a veterinarian environment—for example using a kit for home use.

Suitably, the DNA methylation profile may be provided or determined from a blood sample.

Suitably, the DNA methylation profile may be provided or determined from a hair follicle, buccal swab or saliva sample.

Suitably, the sample may be a blood, buccal swab or saliva sample.

### DNA Methylation

DNA methylation is the process by which a methyl group (CH₃) is added covalently to a cytosine base that is part of a DNA molecule. In vivo, this process is catalysed by a family of DNA methyltransferases (Dnmts), that generate the modified cytosine by transfer of a methyl group from S-adenyl methionine (SAM). The cytosine is modified on the 5^{th} carbon atom, and the modified residue is known as 5-methylcytosine (5mC). The DNA methylation may also comprise 5-hydroxymethylcytosine (5hmc).

DNA methylation is an example of an epigenetic mechanism, i.e. it is capable of modifying gene expression without modification of the underlying DNA sequence. DNA methylation can, for example, inhibit the expression of genes by acting as a recruitment signal for repressive factors, or by directly blocking transcription factor recruitment. DNA methylation predominantly occurs in the genome of somatic mammalian cells at sites of adjacent cytosine and guanine that form a dinucleotide (CpG). While non-CpG methylation is observed in embryonic development, in the adult these modifications are much reduced in most cell types. CpG islands are stretches of DNA that have a high CpG density but are generally unmethylated. These regions are associated with promoter regions, particularly promoter regions of housekeeping genes, and are thought to be maintained in a permissive state to allow gene expression.

The detection of specific methylated DNA can be accomplished by multiple methods (see e.g. Zuo et al., 2009; Epigenomics. 1(2):331-345) and Rauluseviciute et al.; Clinical Epigenetics; 2019; 11(193)). A number of methods are available for detection of differentially methylated DNA at specific loci in samples such as blood, urine, stool or saliva. These methods are able to distinguish 5-methyl cytosine or methylated DNA from unmethylated DNA, and subsequently quantify the proportion of methylated and unmethylated DNA for a particular genomic site.

The present methods may comprise determining a DNA methylation profile for a dog using any suitable method. Suitable methods include, but are not limited to, those described below.

### Enzymatic Methyl-seq (EM-seq)

Suitably, enzymatic approaches are used to detect 5mC and 5hmC. By way of example, Enzymatic Methyl-seq (EM-seq) may be used.

Typically, in EM-seq, in a first enzymatic step, 5mC is oxidized to 5hmC, then 5fC and finally 5caC by the activity of Tet methylcytosine dioxygenase 2 (TET2). In addition, use of a T4-BGT enzyme glucosylates both the pre-existing 5hmC and that produced by TET2 activity. In a second enzymatic step, following denaturation of the double-stranded DNA, the enzyme apolipoprotein B mRNA editing enzyme catalytic polypeptide-like 3A (APOBEC3A) is used to deaminate cytosines, but is unable to deaminate the oxidised or glycosylated forms of 5mC and 5hmC. Only unmethylated cytosines are deaminated to form uracil bases. Prior to the first enzymatic step, the DNA fragments may be generated from mechanical shearing and end-repaired, A-tailed, and ligated to sequencing adaptors, which can be carried out using the NEBNext® DNA Ultra II reagents (NEB), for example. Following the second enzymatic step, the deaminated single-stranded DNA may be amplified by PCR reactions, using polymerase such as NEBNext® Q5U™ which can amplify uracil containing templates, and the resulting library can be sequenced or analysed in an identical manner to the DNA sample generated by bisulfite sequencing. The output of EM-seq is generally the same as whole genome bisulfite sequencing, but with the use of less DNA-damaging reagents, which consequently reduces sample loss, and can outperform bisulfite-conversion prepared samples in coverage, sensitivity and accuracy of cytosine methylation calling. An illustrative EM-seq method is described by Vaisvila et al. (Genome Research; 2021; 31:1-10).

### Bisulfite Conversion-Based Methods

Bisulfite conversion utilizes the selective conversion of unmethylated cytosines to uracil when treated with sodium bisulfite. Denatured DNA is treated with sodium bisulfite, which converts all unmodified cytosines to uracil, and subsequent PCR amplification converts these residues to thymines. Analysing the produced DNA sequences can be done via many different methods, examples of which include but are not limited to: denaturing gel electrophoresis, single-strand conformation polymorphism, melting curves, fluorescent real-time PCR (MethyLight), MALDI mass spectrometry, array hybridization, and sequencing (e.g. Whole Genome Bisulfite Sequencing WGBS). Recently developed techniques such as SeqCap Epi enrich sequences of interest prior to sequencing that enables deeper coverage over a more focused area). Comparison of the abundance of sequences in a bisulfite-converted sample against those of an untreated control allows analysis of methylation at a target site, where the proportion of converted sequences is indicative of the level of methylation at the target site.

Further variants of the bisulfite conversion method are available that are able to distinguish 5mC from the oxidised form 5-hydroxymethylcytosine (5hmC), which behaves identically to 5mC under standard bisulfite conversion, and to detect the further modification 5-formylcytosine (5fC). These methods, such as oxBS-Seq and redBS-Seq, utilise oxidation and reduction of these markers to modify the susceptibility of each species to bisulfite conversion, and through comparative analysis quantify the amount of each modification at target loci.

### Selective Restriction Endonuclease Digestion Methods

Methods of analysing DNA methylation patterns exist may involve the use of restriction enzymes. These include, for example, restriction landmark genomic scanning (RLGS) (Costello et al., 2000; Nat Genet.; 24(2):132-8), methylation-sensitive representational difference analysis (MS-RDA) (Ushijima et al., Proc Natl Acad Sci USA. 1997 Mar. 18; 94(6):2284-9), and differential methylation hybridization (DMH) (Huang et al., Cancer Res. 1997 Mar. 15; 57(6):1030-4). Restriction endonucleases can be methylation dependent in their digestion activity. This specificity can be used to differentiate methylated and unmethylated sequences. Certain restriction enzymes, for example BstUI, HpaII and NotI are sensitive to methylated recognition sequences. Others, such as McrBC, are specific for methylated sequences.

As an example, differential methylation hybridisation (DMH) (Huang et al., as above]) requires an initial fragmentation of the genome with a bulk genome restriction enzyme, such as MseI, which fragments the genome into lengths of less than 200 bp. Following this step, the genome fragments are digested using a methylation-sensitive restriction endonuclease (MREs), or in some versions of the technique, a cocktail of MREs to improve coverage. Depending on the specificity of enzyme or enzymes used, either the methylated or the unmethylated sequences will be degraded. Digested sequences will not be amplified in a subsequent PCR step. The resultant PCR products are suitable for further processing and analysis by sequencing or microarray hybridisation in combination with fluorescent dyes.

Suitably, the present methods utilise a DNA methylation profile generating by a method comprising the use of one or more MREs.

Suitable comparators can be used to investigate methylation state between conditions. DNA from healthy subjects can be compared with aged or diseased subjects to detect changes in methylation state (Huang et al., Hum Mol Genet. 1999 March; 8(3):459-70). Alternatively, a methylation-insensitive version of the secondary digest enzyme, such as the HpaII isoschizomer MspI, can be used to generate a control sample, so that intra- or inter-genomic DNA methylation comparisons can be made (Khulan et al., Genome Res. 2006 August; 16(8):1046-55).

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. Alternatively, the method can encompass amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

Suitably, the digestion of nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization.

Suitable hybridization conditions may be determined based on the melting temperature (Tm) of a nucleic acid duplex comprising the probe. The skilled artisan will be aware that optimum hybridization reaction conditions should be determined empirically for each probe, although some generalities can be applied. Preferably, hybridizations employing short oligonucleotide probes are performed at low to medium stringency. In the case of a GC rich probe or primer or a longer probe or primer a high stringency hybridization and/or wash is preferred. A high stringency is defined herein as being a hybridization and/or wash carried out in about 0.1×SSC buffer and/or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

### Reduced Representation Bisulfite Sequencing (RRBS)

Reduced representation bisulfite sequencing (RRBS) enriches CpG-rich genomic regions using the MspI restriction enzyme-which cuts DNA at all CCGG sites, regardless of their DNA methylation status at the CG site-and enables the measurement of DNA methylation levels at 5%˜10% of all CpG sites in the mammalian genome.

As such, the method involves digestion of DNA using the methylation-insensitive MspI prior the bisulfite conversion and sequencing. Using MspI to digest genomic DNA results in fragments that always start with a C (if the cytosine is methylated) or a T (if a cytosine was not methylated and was converted to a uracil in the bisulfite conversion reaction). This results in a non-random base pair composition. Additionally, the base composition is skewed due to the biased frequencies of C and T within the samples. Various software for alignment and analysis is available, such as Maq, BS Seeker, Bismark or BSMAP. Alignment to a reference genome allows the programs to identify base pairs within the genome that are methylated.

### Affinity Enrichment Based Methods

Distinction of methylated from unmethylated DNA can be accomplished by the use of antibodies, such as anti-5mC, and/or methylated-CpG binding proteins, which contain a methyl-CpG-binding domain (MBD). The antibodies of MBD-domain proteins are able to specifically isolate methylated DNA over unmethylated DNA. Methods that utilize antibodies are commonly referred to as MeDIP, whilst methods utilizing methylated-CpG binding proteins are often known as MBD or MIRA approaches.

These methods require initial fragmentation of the genome, which can be carried out with bulk genome digest with an enzyme such as MseI, which cuts frequently, followed by affinity purification of methylated fragments. The input DNA can be compared to the purified methylated DNA by microarray hybridisation or sequencing to obtain comparative analysis of methylation levels at specific sites.

Further variants of affinity enrichment-based methods are available, such as MethylCap-Seq or MBD-Seq. These methods reduce sample complexity by using a salt gradient to elute methylated DNA fragments in a methy-CpG-abundance dependent manner, segregating CpG islands and other highly methylated loci from less CpG dense loci. The fractions can then be sequenced separately improving sequence coverage.

### Single Molecule Sequencing-Based and De Novo Methylation Sequencing Approaches

Contemporary sequencing methods are able to sequence single molecules directly. Single-molecule real-time (SMRT) DNA sequencing is available, for example the Sequel systems from Pacific Biosciences and has been shown to be able to identify modified bases such as methylated cytosine based on the polymerase kinetics. Nanopore sequencing devices, such as the MinION nanopore sequencer from Oxford Nanopore Technologies, which are able to individually sequence long strands of DNA, are also able to detect de novo base modifications, including methylation.

### DNA Methylation Sites

Suitably, a DNA methylation site may refer to the presence or absence of a 5mC at a single cytosine, suitably a single CpG dinucleotide.

Suitably, a DNA methylation site may refer to the presence or absence of methylation (i.e. the number of 5mC or percentage of 5mC) across a plurality of CpG sites within a DNA region. Suitably, a DNA methylation site may refer to the level of methylation (i.e. the number of 5mC or percentage of 5mC) across a plurality of CpG sites within a DNA region. A “DNA region” may refer to a specific section of genomic DNA. These DNA regions may be specified either by reference to a gene name or a set of chromosomal coordinates. Both the gene names and the chromosomal coordinates would be well known to, and understood by, the person of skill in the art.

Suitably, gene names and/or coordinates may be based on the “Tasha” dog reference genome (https://www.ncbi.nlm.nih.gov/assembly/GCF_000002285.5; Jagannathan et al.; Genes (Basel); 2021; 12(6); 847).

The DNA region may define a section of DNA in proximity to the promoter of a gene, for example. Promoter regions are known to be rich in CpG. By way of example, the DNA region may refer to about 3 kb upstream to about 3 kb downstream; about 2 kb upstream to about 2 kb downstream; about 2 kb upstream to about 1 kb downstream; about 2 kb upstream to about 0.5 kb downstream; about 1 kb upstream to about 0.5 kb downstream; about 0.5 kb upstream to about 0.5 kb downstream of a promoter. Suitably, the DNA region may refer to about 1 kb upstream to about 0.5 kb downstream of a promoter.

Suitably, the DNA region may comprise or consist of CpG sites that are less than about 5000, less than about 4000, less than about 3000, less than about 2000, less than about 1000, less than about 500, or less than about 200 bases apart.

Suitably, the DNA region may comprise or consist of CpG sites that are between about 200 to about 5000, about 200 to about 4000, about 200 to about 3000, about 200 to about 2000, or about 200 to about 1000 bases apart.

Suitably, the DNA region may comprise one or more CpG islands. Suitably, the DNA region may consist of a CpG island.

A “CpG island” may refer to a DNA region comprising at least 200 bp, a GC percentage greater than 50%, and an observed-to-expected CpG ratio greater than 60%.

Suitably, the DNA methylation sites do not comprise X and/or Y chromosome CpGs.

Suitably, the DNA methylation sites do not comprise CpGs known to comprise a SNP at the CpG.

Reference to each of the genes/DNA regions detailed above should be understood as a reference to all forms of these molecules and to fragments or variants thereof. As would be appreciated by the person of skill in the art, some genes are known to exhibit allelic variation between individuals or single nucleotide polymorphisms. Variants include nucleic acid sequences from the same region sharing at least 90%, 95%, 98%, 99% sequence identity i.e. having one or more deletions, additions, substitutions, inverted sequences etc. relative to the DNA regions described herein. Accordingly, the present invention should be understood to extend to such variants which, in terms of the present applications, achieve the same outcome despite the fact that minor genetic variations between the actual nucleic acid sequences may exist between individuals. The present invention should therefore be understood to extend to all forms of DNA which arise from any other mutation, polymorphic or allelic variation.

In terms of screening for the methylation of these gene regions, it should be understood that the assays can be designed to screen for specific DNA. It is well within the skill of the person in the art to choose which strand to analyse and to target that strand based on the chromosomal coordinates. In some circumstances, assays may be established to screen both strands.

“Methylation status” may be understood as a reference to the presence, absence and/or quantity of methylation at a particular nucleotide, or nucleotides, within a DNA region. The methylation status of a particular DNA sequence (e.g. DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.”

Suitably, DNA methylation may be determined using an EM-Seq strategy. In such methods, a methylation level can be determined as the fraction of ‘C’ bases out of ‘C’+‘U’ total bases at a target CpG site “i” following an enzyme and APOBEC3A conversion treatment. In other embodiments, the methylation level can be determined as the fraction of ‘C’ bases out of ‘C’+‘T’ total bases at site “i” following enzyme and APOBEC3A conversion treatment and subsequent nucleic acid amplification. The mean methylation level at each site may then be evaluated to determine if one or more threshold is met.

In some embodiments, in particular when bisulfite conversion and sequencing methods are used, a methylation level can be determined as the fraction of ‘C’ bases out of ‘C’+‘U’ total bases at a target CpG site “i” following a bisulfite treatment. In other embodiments, the methylation level can be determined as the fraction of ‘C’ bases out of ‘C’+‘T’ total bases at site “i” following a bisulfite treatment and subsequent nucleic acid amplification. The mean methylation level at each site may then be evaluated to determine if one or more threshold is met.

Alternatively, a methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of the methylation status. This is of particular use when it is desirable to compare the methylation status of a sequence in a sample to a threshold value.

The present invention is not to be limited by a precise number of methylated residues that are considered to indicative of breed, because some variation between samples will occur. The present invention is also not necessarily limited by positioning of the methylated residue.

In one embodiment, a screening method can be employed which is specifically directed to assessing the methylation status of one or more specific cytosine residues or the corresponding cytosine at position n+1 on the opposite DNA strand.

### Enrichment and Detection Methods

Determining a DNA methylation profile may comprise a step of enriching a DNA sample for selected DNA regions. For example, the methods may comprise a step of enriching a DNA sample for DNA regions comprising the DNA methylation sites which comprise the DNA methylation profile.

Suitable enrichment methods are known in the art and include, for example, amplification or hybridisation-based methods. Amplification enrichment typically refers to e.g. PCR based enrichment using primers against the DNA regions to be enriched. Any suitable amplification format may be used, such as, for example, polymerase chain reaction (PCR), rolling circle amplification (RCA), inverse polymerase chain reaction (pCR), in situ PCR, strand displacement amplification, or cycling probe technology.

Hybridisation enrichment or capture-based enrichment typically refers to the use of hybridisation probes (or capture probes) that hybridise to DNA regions to be enriched.

The hybridisation probe(s) may be attached directly to a solid support, or may comprise a moiety, e.g. biotin, to allow binding to a solid support suitable for capturing biotin moieties (e.g. beads coated with streptavidin). In any case, DNA comprising sequence which is complementary to the probe may be captured thus allowing to separate DNA comprising DNA regions of interest from not comprising the DNA regions of interest. Hence, such a capturing steps allows to enrich for the DNA regions of interest. For example, the DNA regions may be DNA regions in proximity to gene promoters.

An array used herein can vary depending on the probe composition and desired use of the array. For example, the nucleic acids (or CpG sites) detected in an array can be at least 10, 100, 1,000, 10,000, 0.1 million, 1 million, 10 million, 100 million or more. Alternatively or additionally, the nucleic acids (or CpG sites) detected can be selected to be no more than 100 million, 10 million, 1 million, 0.1 million, 10,000, 1, 000, 100 or less. Similar ranges can be achieved using nucleic acid sequencing approaches such as those known in the art; e.g. Next Generation or massively parallel sequencing.

Suitably, an enrichment step may be performed before or after the step of separating or differentially treating methylated and unmethylated DNA.

As used herein, the term “enriching” or “enrichment” for “DNA” or “DNA regions” means a process by which the (absolute) amount and/or proportion of the DNA comprising the desired sequence(s) is increased compared to the amount and/or proportion of DNA comprising the desired sequence(s) in the starting material. In this regard, enrichment by amplification increases the amount and proportion of the desired sequence(s). Enrichment by capture-based enrichment increases the proportion of DNA comprising the desired sequence(s).

Following processing of the DNA to distinguish methylated and unmethylated sites, the present methods may further comprise the step of identifying the sites which were methylated or unmethylated (i.e. in the original sample).

The identification step may comprise any suitable method known in the art, for example array detection or sequencing (e.g. next generation sequencing).

A sequencing identification step preferably comprises next generation sequencing (massively parallel or high throughput sequencing). Next generation sequencing methods are well known in the art, and in principle, any method may be contemplated to be used in the invention. Next generation sequencing technologies may be performed according to the manufacturer's instructions (as e.g. provided by Roche, lllumina, Applied Biosystems, PacBio, Oxford Nanopore or MGI).

In a preferred embodiment, the sample is treated by converting DNA methylation using enzymatic reactions, performing whole genome library preparation and measuring the methylation profile by sequencing (EM-Seq).

In a particularly preferred embodiment, the sample is treated by converting DNA methylation using enzymatic reactions, performing whole genome library preparation, hybridizing the whole-genome-converted library preparation to capture probes (preferably capture probes capable of capturing DNA regions in proximity to gene promoters); and measuring the methylation profile by sequencing (EM-Seq).

### DNA Methylation Profile

A “DNA methylation profile” or “methylation profile” may refer to the presence, absence, quantity or level of 5mC at one or more DNA methylation sites. Preferably, “methylation profile” refers to the presence, absence, quantity or level of 5mC at a plurality of DNA methylation sites. Thus, the presence, absence, quantity or level of 5mC at each individual DNA methylation site within the plurality of sites may be assessed and contribute to the determination of whether a dog has, or is at risk of developing, OA. The quality and/or the power of the methods may thus be improved by combining values from multiple DNA methylation markers.

Suitably, the present DNA methylation comprises the methylation profile from a plurality of methylation sites.

Suitably, presence or absence of 5mC from at least 5, at least 10, at least 20, at least 50, at least 100, at least 200, at least 500, at least 1000, at least 2000, at least 5000, at least 10000, at least 50000, at least 10000, at least 250000, or at least 500000 DNA methylation sites may be used to determine the risk of developing OA.

Suitably, the methylation profile may refer to the presence or absence of 5mC from at least 100, at least 200, at least 500, at least 1000 or at least 2000 DNA methylation sites.

Suitably, the methylation profile may refer to the presence or absence of 5mC from at least 5, at least 10, at least 20, at least 50, at least 100, or at least 200 methylation sites.

Suitably, the methylation profile may refer to the presence or absence of 5mC from about 5, about 10, about 20, about 50, about 100, about 200, about 500, about 1000 or about 2000 DNA methylation sites.

Suitably, the methylation profile may refer to the presence or absence of 5mC from about 100, about 200, about 500, about 1000 or about 2000 DNA methylation sites.

Suitably, the methylation profile may refer to the presence or absence of 5mC from about 5, about 10, about 20, about 50, about 100, or about 200 DNA methylation sites.

Suitably, the methylation profile may refer to the presence or absence of 5mC from about 5, about 10, about 20, about 40, or about 100 DNA methylation sites.

In order to generate a profile for determining OA, or a risk of developing OA, an initial methylation profile may be processed or streamlined to produce a restricted methylation profile which is then used to determine OA, or a risk of developing OA. A restricted methylation profile may also be produced which is associated with a dog that is known to have developed OA or known not to have developed OA.

By way of example, an initial methylation profile may be processed or streamlined by—for example—using DNA regions rather than individual cytosines, by selecting a subset of methylation sites that are associated with a particular physiological or biochemical pathway, performing a correlation analysis and retaining one or more representative DNA methylation sites per cluster, or performing differential analysis to pre-select DNA methylation sites or retain DNA methylation sites that vary more between dogs that have OA or do not have OA. The processing of the DNA methylation sites may comprise calculating the mutual information for each of the sites, ordering them according to this metric and then selecting the top-n to put in the model.

For example, the DNA region(s) may be any DNA region(s) as defined herein.

Suitably, the methylation profile may refer to DNA methylation sites of genes that are associated with a particular physiological or biochemical pathway. As such, the methylation profile may comprise methylation sites associated with a particular tissue, organ, or physiological system. Determining the status methylation sites associated with a particular tissue, organ or physiological system may advantageously allow the method to be utilised in a way which focuses on pathologies and diseases of that tissue, organ or physiological system.

In some embodiments, a threshold selects those sites having the highest-ranked mean methylation values for determining an OA risk. For example, the threshold can be those sites having a mean methylation level that is the top 50%, the top 40%, the top 30%, the top 20%, the top 10%, the top 5%, the top 4%, the top 3%, the top 2%, or the top 1% of mean methylation levels across all sites “i” tested for a predictor, e.g., OA occurrence.

Alternatively, the threshold can be those sites having a mean methylation level that is at a percentile rank greater than or equivalent to 50, 60, 70, 80, 90, 95, 96, 97, 98, or 99. In other embodiments, a threshold can be based on the absolute value of the mean methylation level. For instance, the threshold can be those sites having a mean methylation level that is greater than 99%, greater than 98%, greater than 97%, greater than 96%, greater than 95%, greater than 90%, greater than 80%, greater than 70%, greater than 60%, greater than 50%, greater than 40%, greater than 30%, greater than 20%, greater than 10%, greater than 9%, greater than 8%, greater than 7%, greater than 6%, greater than 5%, greater than 4%, greater than 3%, or greater than 2%. The relative and absolute thresholds can be applied to the mean methylation level at each site “i” individually or in combination. As an illustration of a combined threshold application, one may select a subset of sites that are in the top 3% of all sites tested by mean methylation level and also have an absolute mean methylation level of greater than 6%. The result of this selection process is a DNA methylation profile, of specific hypermethylated sites (e.g., CpG sites) that are considered the most informative for OA occurrence.

Suitably, the DNA methylation profile may comprise at least one methylation site as listed in Table 1.

Suitably, the methylation site(s) may be defined as the methylation markers present in any one or more of SEQ ID NO: 1-90. SEQ ID NO: 1-90 show the sequence either side of the methylation marker in the “Tasha” dog reference genome (https://www.ncbi.nlm.nih.gov/assembly/GCF_000002285.5; Jagannathan et al.; Genes (Bsael); 2021; 12(6); 847). The “CG” methylation marker are the 26^{th} and 27^{th} nucleotides in the sequence (i.e. there are 25 nucleotides preceding the methylation marker and 25 nucleotides following the methylation marker).

Suitably, the methylation site may be defined as the intervening position in the column labelled “Site” in Table 1. For example, for site chr1: 107909097-107909099, the methylation marker is chr1: 107909098.

Suitably, the DNA methylation profile may comprise at least one methylation site as listed in Table 1.

Suitably, the DNA methylation profile may comprise at least 2, at least 5, at least 10, at least 25, at least 50, at least 60, at least 70, at least 80 or each of the methylation sites as listed in Table 1.

Suitably, the DNA methylation profile may comprise the methylation sites listed as site numbers 1 to 5 in Table 1.

Suitably, the DNA methylation profile may comprise the methylation sites listed as site numbers 1 to 10 in Table 1.

Suitably, the DNA methylation profile may comprise the methylation sites listed as site numbers 1 to 25 in Table 1.

Suitably, the DNA methylation profile may comprise the methylation sites listed as site numbers 1 to 50 in Table 1.

Suitably, the DNA methylation profile may comprise the methylation sites listed as site numbers 1 to 60 in Table 1.

Suitably, the DNA methylation profile may comprise the methylation sites listed as site numbers 1 to 70 in Table 1.

Suitably, the DNA methylation profile may comprise the methylation sites listed as site numbers 1 to 80 in Table 1.

Suitably the DNA methylation profile may comprise at least 5 of the methylation sites as listed in Table 1, wherein at least 4 of the methylation sites comprise the methylation sites listed as site numbers 1 to 4 in Table 1.

Suitably the DNA methylation profile may comprise at least 10 of the methylation sites as listed in Table 1, wherein at least 5 of the methylation sites comprise the methylation sites listed as site numbers 1 to 5 in Table 1. Suitably the DNA methylation profile may comprise at least 10 of the methylation sites as listed in Table 1, wherein at least 9 of the methylation sites comprise the methylation sites listed as site numbers 1 to 9 in Table 1.

Suitably the DNA methylation profile may comprise at least 25 of the methylation sites as listed in Table 1, wherein at least 10 of the methylation sites comprise the methylation sites listed as site numbers 1 to 10 in Table 1. Suitably the DNA methylation profile may comprise at least 25 of the methylation sites as listed in Table 1, wherein at least 20 of the methylation sites comprise the methylation sites listed as site numbers 1 to 20 in Table 1.

Suitably the DNA methylation profile may comprise at least 50 of the methylation sites as listed in Table 1, wherein at least 10 of the methylation sites comprise the methylation sites listed as site numbers 1 to 10 in Table 1. Suitably the DNA methylation profile may comprise at least 50 of the methylation sites as listed in Table 1, wherein at least 25 of the methylation sites comprise the methylation sites listed as site numbers 1 to 25 in Table 1.

Suitably, the DNA methylation profile may comprise each of the methylation sites listed in Table 1.

### Determination of DNA Methylation Profiles Indicative of OA

The present invention comprises utilising a DNA methylation profile to determine whether a canine subject has OA, or is at risk developing OA.

Suitably, determining a ‘risk of developing OA’ may comprise determining a predicted probability of a dog belonging to an OA class or non-OA class (e.g. based on a methylation profile determined from a model as described herein).

The provision of a DNA methylation profile that is indicative of OA, or a risk of a canine subject developing OA, may be achieved through training datasets and machine learning approaches, for example. Suitably, the machine learning approaches may be supervised machine learning approaches. Suitably, the model may be a multinomial regression, support-vector machine (SVM) or a random forest model.

By way of example, DNA methylation sites (or a DNA methylation profile) may be trained against a dataset comprising dogs that were known to have OA or not have OA. Suitably, DNA methylation sites (or a DNA methylation profile) may be trained against a dataset comprising dogs that were known to subsequently develop OA or not develop OA after the samples from which the dataset was generated were taken.

Suitably, whether a dog has, or does not have, OA or whether a dog was known to develop, or not to develop, OA may be referred to as the OA status of the dog.

Suitably, the DNA methylation sites (or a DNA methylation profile) may be trained against a dataset comprising dogs of a known OA status in combination with known age and/or sex.

For example, models for DNA methylation sites (or a DNA methylation profile) indicative of OA, or OA risk, may be provided by training a dataset of methylation status at a plurality of DNA methylation sites against a training dataset of dogs with a known OA status using a machine learning framework, and testing against a with-held cohort to validate the veracity of the model.

The machine learning framework may comprise fitting a penalised regression to a training dataset of dogs with a known OA status (and optionally age and/or sex); for example, using glmnet R package.

The machine learning framework may comprise fitting a multinomial model, a random forest, a SVM (support vector machine), penalized multinomial logistic regression, on the features or PC (principal component) transformed features or other model used to predict multi class outcomes.

Suitably, the machine learning framework may comprise fitting a penalised regression, such as an elastic net regression, of OA status explained by a DNA methylation profile and/or genetic markers, (and optionally age and/or sex).

Suitably, the machine learning framework may comprise fitting a penalised regression, such as an elastic net regression, of OA status explained by a DNA methylation profile, age and sex.

Suitably, the machine learning framework may be used to determine a model comprising a set of DNA methylation sites (or a DNA methylation profile) that is indicative of OA status.

Suitably, the machine learning framework may be used to determine a model comprising a set of DNA methylation sites (or a DNA methylation profile) that is indicative of OA status.

The model may comprise the methylation status at a plurality of DNA methylation sites; wherein the methylation status at each site is considered in the model by multiplying by a coefficient value.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values.

Suitably, a probability of developing OA may be determined by fitting a logistic regression to the methylation profile and calculating:

$$P = \frac{1}{1 + {e}^{- ( {β}_{0} + {β}_{1} {x}_{1} + … + {β}_{10} {x}_{10} )}}$$

Where βᵢ are the coefficients provided herein (e.g. as provided in Table 3) and xᵢ represents the methylation level of site i.

Suitably, sex may be coded as a numerical value with 0 for female and 1 for male.

Suitably, the machine learning platform may comprise one or more deep neural networks. Neural Networks are collections of neurons (also called units) connected in an acyclic graph. Neural Network models are often organized into distinct layers of neurons. For most neural networks, the most common layer type is the fully-connected layer in which neurons between two adjacent layers are fully pairwise connected, but neurons within a single layer share no connections. One of the main features of deep neural networks is that neurons are controlled by non-linear activation functions. This non-linearity combined with the deep architecture make possible more complex combinations of the input features leading ultimately to a wider understanding of the relationships between them and as a result to a more reliable final output. Deep neural networks have been applied for many types of data ranging from structural data to chemical descriptors or transcriptomics data.

Suitably, the machine learning platform comprises one or generative adversarial networks. Suitably, the machine learning platform comprises an adversarial autoencoder architecture. Suitably, the machine learning platform comprises a feature importance analysis for ranking DNA methylation sites and/or genetic markers by their importance in determining OA status.

### Comparison to a Reference or Control

The present method may comprise a step of comparing the difference in DNA methylation at one or more sites in the test sample to one or more reference or controls. The presence or absence of DNA methylation at one or more sites in the reference or control may be associated with OA status or a risk of developing OA. In some embodiments, the reference value is a value obtained previously for a subject or group of subjects with a known OA status. The reference value may be based on a known DNA methylation status at one or more sites, e.g. a mean or median level, from a group of subjects with known OA status. The reference DNA methylation profiles may comprise DNA methylation profiles from at least 1, at least 2, at least 4, at least 10, at least 20, at least 40, at least 80, at least 100, at least 150, at least 200, at least 300, at least 400 dogs, at least 500, at least 750 or at least 1000 dogs.

### Osteoarthritis (OA)

Osteoarthritis (OA) is a chronic and progressive joint disease that commonly affects dogs. This degenerative condition makes dogs lose their joint cartilage, leading to other changes in their bones and causing pain, inflammation and difficulty using the affected limb.

OA is not just a disease that occurs in senior dogs and can develop at any age, especially in dogs with contributing factors, such as obesity or other orthopedic conditions.

It is to be appreciated that all references herein to treatment include curative, palliative and prophylactic treatment, although in the context of the invention references to preventing are more commonly associated with prophylactic treatment. Treatment may also include arresting progression in the severity of a disease.

### Method for Selecting a Dietary, Pharmacological, or Lifestyle Regime for a Dog

In a further aspect, the present invention provides a method for selecting a dietary, pharmacological, or lifestyle regime for a subject.

Illustrative examples of a dietary regime that may be applied to a dog with OA, or at risk of OA, include calorie-restricted (‘weight-loss’) diets.

By the term “dietary intervention” it is meant an external factor applied to a subject which causes a change in the subject's diet. More preferably the dietary intervention includes the administration of at least dietary product or dietary regimen or a nutritional supplement.

The dietary regime may be a meal, a regime of meals, a supplement or a regime of supplements, or combinations of a meal and a supplement, or combinations of a meal and multiple supplements.

The dietary intervention or dietary product described herein may be any suitable dietary regime, for example, a calorie-restricted diet. A dietary intervention may be determined based on the baseline maintenance energy requirement (MER) of the dog or dog breed. Suitably, a calorie-restricted diet may comprise about 50%, about 55%, about 60%, about 65%, about 75%, about 80%, about 85%, or about 90% of the dog's MER. Suitably, a calorie-restricted diet may comprise about 60% or about 75% of the dog's MER.

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

The dietary regime may also comprise glucosamine, chondroitin, long-chain omega-3 fatty acids (e.g. eicosapentaenoic acid), and/or polysulfated glycosaminoglycan supplementation.

The dietary, pharmacological, or lifestyle regime may be applied to the dog for any suitable period of time. By way of example, the dietary, pharmacological, or lifestyle regime may be applied for at least 2, at least 4, at least 8, at least 16, at least 32, or at least 64 weeks. The dietary, pharmacological, or lifestyle regime may be applied for at least 3, at least 6, at least 12, at least 24, at least 36, at least 48 or at least 60 months. Suitably, the dietary, pharmacological, or lifestyle regime may be applied for at least 1, at least 2, at least 3, at least 4, at least 5, at least 6, at least 7, at least 8, at least 9 or at least 10 years. Suitably, the dietary, pharmacological, or lifestyle regime may be applied for the lifetime of the dog.

The modification in lifestyle may be any change as described herein, e.g. a change in exercise regime. For example, the modification in lifestyle may comprise passive range-of-motion exercises, and/or weight loss and exercise regimes.

Ideal activity level and type may differ according to breed or breed categorisation. For example, a robust dog will be spontaneously engaged in mild (e.g., slow walking), moderate (e.g., brisk walking) or occasionally intense (e.g., running) activity types. An athletic dog, in comparison, will mainly be voluntary involved in moderate, intense or very intense (e.g., fast running) activities. Within these different levels of activity, dogs can be further classified as robust or athletic.

The pharmacological regime may refer to administration of a therapeutic modality or regimen. The pharmacological regime may be non-steroidal anti-inflammatory drugs. The present invention may thus advantageously enable the identification of dogs that are expected to respond particularly well to a given intervention (e.g. dietary, pharmacological or lifestyle regime). The intervention can thus be applied in a more targeted manner to dogs that are expected to respond due OA status.

### Use of a Dietary Intervention

In one aspect, the present invention provides a dietary or pharmacological intervention for use in reducing the risk that a canine subject will develop OA, wherein the dietary or pharmaceutical product is administered to a canine subject that has been determined to be at risk of developing OA according to the present invention.

In one aspect, the present invention provides a dietary or pharmacological intervention for use in treating and/or preventing OA in a canine subject, wherein the dietary intervention is administered to a canine subject that has been determined have OA, or be at risk of developing OA, by the present method.

As described herein, the dietary intervention may be a dietary product or dietary regimen or a nutritional supplement.

### Computer Program Product

The present methods may be performed using a computer. Accordingly, the present methods may be performed in silico.

Suitably, the computer may prepare and share a report detailing the outcome of the present methods.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine whether a canine subject has or is at a risk of developing osteoarthritis (OA) as described herein.

In another aspect, the present invention provides a computer program product comprising computer implementable instructions for causing a device to determine whether a canine subject has or is at a risk of developing osteoarthritis (OA); and optionally select a suitable dietary, pharmacological, or lifestyle regime for the dog based whether the canine subject has, or is at risk of developing OA, as determined by the present method. The computer program product may also be given additional parameters or characteristics for the dog. As described herein, the additional parameters or characteristics may include age and sex of the dog.

In one embodiment, the user inputs into the device levels of one or more of DNA methylation markers, optionally along with age and sex. The device then processes this information and provides a determination of whether a canine subject has or is at a risk of developing osteoarthritis (OA). Alternatively, the device then processes this information and provides a determination of a suitable dietary, pharmacological, or lifestyle regime for the dog based on whether the canine subject has, or is at risk of developing OA, as determined by the present method.

The device may generally be a server on a network. However, any device may be used as long as it can process biomarker data and/or additional parameters or characteristic data using a processor, a central processing unit (CPU) or the like. The device may, for example, be a smartphone, a tablet terminal or a personal computer and output information indicating the determined OA status for the dog or a determination of a suitable lifestyle or dietary regime for the dog based on the OA status.

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

All publications mentioned in the specification are herein incorporated by reference.

### EXAMPLES

The invention will now be further described by way of examples, which are meant to serve to assist one of ordinary skill in the art in carrying out the invention and are not intended in any way to limit the scope of the invention.

### Example 1—Illustrative Method for Determining Osteoarthritis Risk in Dog Using DNA Methylation

### Dataset Description

An osteoarthritis (OA) risk assessment tool using DNA methylation data was developed using a pet cohort composed of 829 dogs and 20 breeds. The training cohort was split into two subsets: training and testing sets. The training set comprised of dogs that potentially shared a familial relationship, while the testing set contained dogs unrelated to those in the training set.

Methylation Data (Tasha, canFam6)

Using the blood samples from the pet cohort, the methylation beta-values at sites near promoters were obtained by sequencing using Enzymatic Methyl-seq (EMSeq). EMSeq was analyzed mapping to the canFam6 genome assembly (also referred to as Tasha). The lowly covered (<15) and missing values were first imputed using the Boostme algorithm (Zou et al. BMC Genomics 19, 390 (2018)), a tree-based machine learning algorithm.

The X chromosome was removed. The sites were selected to reduce correlations and therefore dimension of the features space using EBmodule (Zollinger et al.; Biostatistics; 19(2); 153-168; 2018). The dataset was separated into blocks of about 5000 sites by grouping the sites per target (1500 bp around the TSS) and per chromosome. A correlation matrix was calculated on which EB modules as was applied described in (Zollinger et al.; supra.). Each module was then represented by the medoid site. Some sites did not belong to any modules and were called scattered sites (defined as per Zollinger et al.; supra.). The number of sites was reduced to 471K from 1.4 mio.

### OA Risk Estimation Using DNA Methylation

To reduce the dimensionality of the DNA methylation data, an epigenome association study (EWAS) to find the CpGs associated to OA was performed. To mitigate the potential confounding effect of breed-associated methylation sites and focus on sites directly linked to the disease, the EWAS was conducted on a subset of the data consisting only of the Labradors from the training set, the Labrador breed having the highest OA incidence.

To prevent overfitting, a repeated cross-validation framework was employed. The training cohort was divided into 10 folds, with EWAS performed on all folds except one. This process was repeated for the 10 folds, and the entire procedure was repeated 10 times using different folds. The p-value for each repetition was recorded and averaged.

A logistic regression was then trained with lasso regularization (lambda=0.05) starting from all the CpGs with an average p-value less than 0.1 in the EWAS results. The final model used 90 methylation sites (see Table 1).

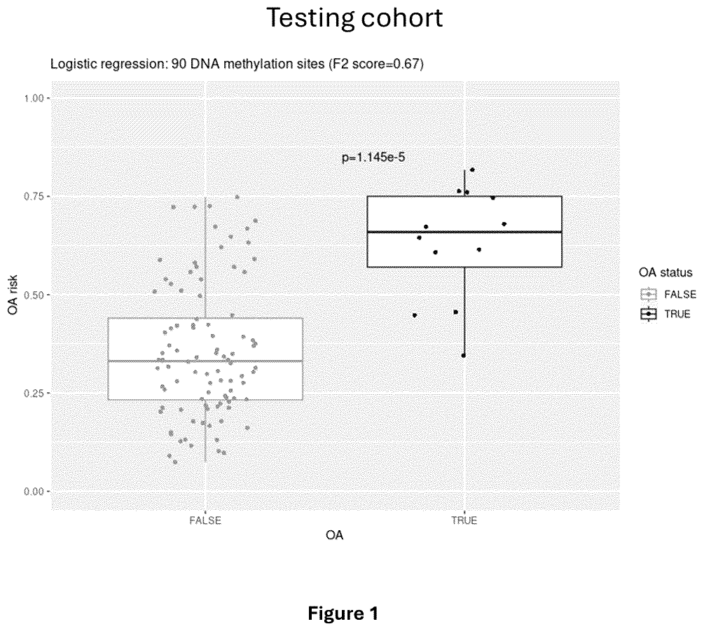

To address the imbalanced distribution of OA cases, different weights were assigned to the control and case dogs. The weight for control dogs was defined as the fraction of OA cases in the training set, while the weight for case dogs was defined as one minus this fraction. The 90 methylation site model achieved a F2 score of 0.67 in the testing set (see FIG. 1).

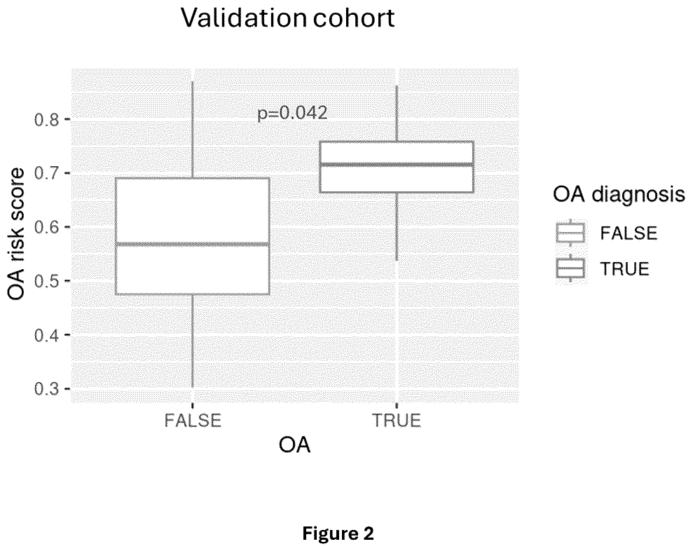

Finally, to assess the generalizability of the model, we used a validation cohort, composed of Labradors Retrievers. The model predicts a significantly higher risk for dogs in the validation cohort that developed OA (TRUE) compared to dogs that did not develop OA (FALSE) (see FIG. 2). Further classification models were also generated using only the top 10, top 20, and top 50 sites from the complete list of sites shown in Table 1; and each was shown to be predictive of OA risk (see Table 2). These classifiers were generated by selecting the top-n sites based on mutual information going from the most informative (top of the list) to the least informative. Average F2 score for the classifier using the top 5, top 10, top 20, top 50, and top 90 sites are shown in Table 2.

TABLE 2
F2 training cross-validation on different numbers of sites

| Number of sites | F2_train_cross_validated |
|---|---|
| 5 | 0.4666667 |
| 10 | 0.5612245 |
| 20 | 0.5625 |
| 50 | 0.5263158 |
| 90 | 0.67 |

### Description of Coefficients Using Logistic Regression

To provide coefficients for a model concisely, logistic regression (LR) was fitted with the same parameters, but only with the top 10 methylation sites. The coefficients for this model are given for the 10 markers, in addition to the intercept, in Table 3.

To calculate the probability/risk of OA, one should calculate:

$$P = \frac{1}{1 + {e}^{- ( {β}_{0} + {β}_{1} {x}_{1} + … + {β}_{10} {x}_{10} )}}$$

Where βᵢ are the coefficients provided in Table 3 and xᵢ represents the methylation level of site i.

TABLE 3

| Site | | |
|---|---|---|
| Number | Site | Coefficient |
| Intercept | N/A | −1.14 |
| 1 | chr1.107909097.107909099 | −10.35 |
| 2 | chr2.51404290.51404292 | −14.01 |
| 3 | chr38.14762793.14762795 | 9.34 |
| 4 | chr4.28931188.28931190 | −7.13 |
| 5 | chr17.21218848.21218850 | −4.82 |
| 6 | chr35.7689768.7689770 | 8.07 |
| 7 | chr15.10375805.10375807 | 3.22 |
| 8 | chr5.56574849.56574851 | −6.12 |
| 9 | chr20.32110707.32110709 | −8.05 |
| 10 | chr20.44395099.44395101 | 3.65 |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1
Methylation sites associated with OA risk

| Site | | Sequence (25 bases upstream and downstream | SEQ |
|---|---|---|---|
| Number | Site | of the 5mC site) | ID NO: |
| 1 | chr1.107909097.107909099 | GCTCCGGGTTCGGGGGGGGCGGGAGGCGAAAGGGAT | 1 |
| | | CCGCGCTCCCAGACCCC | |
| 2 | chr2.51404290.51404292 | CCACCTGTCCGGGCCTTTCCGGGGTGCGTGGGCGGCG | 2 |
| | | CCTCGGCGGGCGGAGC | |
| 3 | chr38.14762793.14762795 | CCGCGCCAGGCGCAGCACGGCCTCCACGGCGATGGT | 3 |
| | | GAAGCAGAGCGCGCTGA | |
| 4 | chr4.28931188.28931190 | CCCCGGGGGCGGTGGTGCTTCCTCTGCGCGCCCCGCC | 4 |
| | | AGTCGGTGCCCGGGCT | |
| 5 | chr17.21218848.21218850 | GTTACTCAGGCGTAGCTTGTGCCAGACGACTTCTGGC | 5 |
| | | CAGCGTTTTCAAGGGG | |
| 6 | chr35.7689768.7689770 | GCGGCGGTGCCGAGAGTCGAGGGGCGCGGAGTCTGC | 6 |
| | | GAGCGGGGAAGTTCAGC | |
| 7 | chr15.10375805.10375807 | GTGGCTGGAGCCGCGCGAGCCAGAACCGCGGGGGCA | 7 |
| | | GGTCCTCGCAGGGCCCG | |
| 8 | chr5.56574849.56574851 | GGGCGCCTTCTTCTCCCGGGGCCTTCCGCCTGCTATCA | 8 |
| | | GCTGTTAAGGCTGGG | |
| 9 | chr20.32110707.32110709 | CCTCCGCGTGGTCTCTGCCCGCCTTGCGCCCCCTCTCA | 9 |
| | | GCGTCCTCTCTCTCT | |
| 10 | chr20.44395099.44395101 | CAGTTATCTGAAAAGTTTCTGGCCGCCGGGAGAGGAT | 10 |
| | | GAATTTCAAGGTGGCA | |
| 11 | chr5.58377390.58377392 | GGGACGCCTCCCAGAGTTTAGACGGACGAACCCAGG | 11 |
| | | CTCGCTAGGACGCCCAG | |
| 12 | chr5.8362309.8362311 | CCAACGTGCTTGCCAAGGCGCTGAGCCGCGGCGTTCT | 12 |
| | | GCACCAAGACAAGGAT | |
| 13 | chr3.15322082.15322084 | TGGAGGGGCCGAAGGGGTGCTCCATCCGCGGCCCGC | 13 |
| | | GGCGGGGAAGGGGGTCC | |
| 14 | chr3.58007517.58007519 | GGCGCCGGGTGCTGCCGGAGGGGCGGCGGGGGGCGC | 14 |
| | | CCGGGCCGCTGGCGAGG | |
| 15 | chr20.40194406.40194408 | ACTCCTACTATCTACCAGGCCTAACCCGTTTGACATA | 15 |
| | | TCACAGGCCCCCTGCC | |
| 16 | chr2.9497618.9497620 | GGCGTCCGCGGGGAGCCGGGGCTCTTCGCCGGCCGG | 16 |
| | | ACCCACACCCGCCGGCT | |
| 17 | chr24.24891299.24891301 | CCGCCCAGCGCCGCCCGGAGCAGCCGCGGCCCCGCG | 17 |
| | | AGGAGACGGGCGCCGCC | |
| 18 | chr6.50215959.50215961 | CCTTCCGGAGCAAGGGAGCCCCGAGTCGCGGGTCCC | 18 |
| | | ACGCCCGCCACCCGAGG | |
| 19 | chr24.36179043.36179045 | GACAGTACCCACGAAAAGGGGGCAGACGTTTGGCCT | 19 |
| | | GAGGGGCAGCAGGCACG | |
| 20 | chr17.24850422.24850424 | GCCCGCGGCCCCGCGCGGCCAGCCCTCGCTCGGCTCA | 20 |
| | | GCGTCGATGCCCCTGT | |
| 21 | chr9.4872022.4872024 | TCCCCGCCGCGTGCACGGTGCGCTCCCGCCCAGCGCC | 21 |
| | | CGACCCCGCCCGCCGC | |
| 22 | chr9.6208373.6208375 | CTAGGATCCAGACTGCCCGCAGACACCGCTGCCCAGT | 22 |
| | | CCTTGGCCCCTTCCTT | |
| 23 | chr5.60094293.60094295 | GGCAGGCGGGGGGCGGCACACATGCCCGGATTGTGA | 23 |
| | | CGTCCAGTCTGTTGCTG | |
| 24 | chr32.28602756.28602758 | GGCGGGGGCGAGAGGCGGTCCCGGCTCGGGCACTGC | 24 |
| | | GGCTCCGCCGGGAGCCC | |
| 25 | chr8.26049369.26049371 | CCCGACCGGGCTGGTGACCCTGAGGCCGGGTGCCTGG | 25 |
| | | CGGTCTGTACCCTGCA | |
| 26 | chr18.48968531.48968533 | AACCGGTGAGAAAGCAGGTGGAACAGCGGGAGGTTT | 26 |
| | | CCCCTTCTCTGAATGAA | |
| 27 | chr25.17111544.17111546 | TGAACAGCAGGACGAGAACTTTGTGGCGTAGCGTGA | 27 |
| | | ACGCGCTTCAGGATCAG | |
| 28 | chr21.40486217.40486219 | TGTGATTTACTTCCCTGCTTTATCCCCGACTTGAGCCA | 28 |
| | | CTGGGCTTAAAAAAG | |
| 29 | chr14.21632844.21632846 | CCCGCAGCCCCGTGCGCGGAGCCGCGCGGCCCCCGG | 29 |
| | | CTGCAGCTCCGGGCTGG | |
| 30 | chr20.44094123.44094125 | ACCTACTGTGTGCAAGCACCCAGCTCCGTGCGATAGA | 30 |
| | | GGAGCCCCGGCCTCTA | |
| 31 | chr26.8570602.8570604 | TGTCCCTTTCATACGGAAGGGAAAACCGAGGCCCAA | 31 |
| | | GAGAGCGTAGGTGGGAC | |
| 32 | chr24.32820087.32820089 | TTTTCGTGCGAGGGGGCCTCCAGCCGCGCTCAGCCCG | 32 |
| | | CCCCTCGTCTCCTCTT | |
| 33 | chr12.665279.665281 | GAGGCAGTCCAGGCCTTGTTAGGTAGCGCTGGGAATC | 33 |
| | | ACAAGTGGGGTGGCTT | |
| 34 | chr4.39797843.39797845 | CCACGCGTCTTCACGCGGCAGGCAGGCGTTCCAGGCC | 34 |
| | | CATTTCACAGATGAAG | |
| 35 | chr20.3126525.3126527 | AGTCAGAGGGCTGCTTGAGGGACTCTCGCTGGTAAGA | 35 |
| | | AGTCGATCTGAAACAG | |
| 36 | chr4.50543804.50543806 | CTCAAGAGTAAGTTTCTGATGGAGCTCGGAACCCTGA | 36 |
| | | GGGTCAACTTTACCCA | |
| 37 | chr29.5921980.5921982 | CAGCCCCGCCGCCGGCTGGGGCCCTCCGGGAGCCCG | 37 |
| | | GGGCGACCCCAGCCCCC | |
| 38 | chr10.7101684.7101686 | CCGCAGCGCAGTCCGCGGGCAGGAGGCGTCACCCGA | 38 |
| | | AGGAACAGCCGCGAGGA | |
| 39 | chr6.41679153.41679155 | TTTCTTACTTCTATGCTAGGGTTTCCCGGTTGCACCTA | 39 |
| | | CATTTTAAAAGTGTT | |
| 40 | chr20.52410067.52410069 | TGTGCCACAGAGGGGTCCGCAGGCGTCGGACAGACA | 40 |
| | | CGGGCCGAGGAGAATTC | |
| 41 | chr28.10279632.10279634 | TGTTTGAGCTGAAGAAACGAGCCAAACGCCTCGCTGG | 41 |
| | | GATGCCCACAGGGAGT | |
| 42 | chr21.23435769.23435771 | TCTGGTTCCCCTTGGACTCCCACGCTCGAGCCCGTAA | 42 |
| | | GTAGGCAGAGGAGGGA | |
| 43 | chr4.20970595.20970597 | GAGGGGATGGGACCGAGGCACGTGGACGTGGGAGCA | 43 |
| | | ATAGTGACCCAAGGGAT | |
| 44 | chr7.74853992.74853994 | GCCCTTGCCAATTCAGGAATTCTCGGCGGGGTCTGCA | 44 |
| | | CACACAGTTTCACGCA | |
| 45 | chr9.47924936.47924938 | GACAGATCCGCTGCCCTCACAGTAACCGGGCTGCCAG | 45 |
| | | AGCTGCTGACCCGGCC | |
| 46 | chr16.15250294.15250296 | CCATAACTGATGCCCACAGGGTCTGACGTTCTGAGTT | 46 |
| | | AACCTGCATTATAACT | |
| 47 | chr5.77139699.77139701 | CCGTTCTTCTGCTGCGGAGGGTCGCACGAGGCCTCGA | 47 |
| | | ACACGGCGAGGCGCGG | |
| 48 | chr38.1909686.1909688 | GTCGGCCCGGGGCCGGGCAAGCTCGGCGGCCAGCGC | 48 |
| | | GGAGGCGGCGGCGGGAG | |
| 49 | chr16.45892065.45892067 | GAGCGGCCGCCAGTGGCCGCGGCCGGCGCGCGGTGG | 49 |
| | | AGGCCGGCGCCGCTGCT | |
| 50 | chr10.19454770.19454772 | CGCTGCTGGCTCAGCCCTAGGTCTTCCGCCTCTGTAA | 50 |
| | | ATGTTTGTTGGAGGGA | |
| 51 | chr8.49821467.49821469 | TACAAACACCCAGAAACGCTGGGGGGGGGGGGAGGG | 51 |
| | | GCACGGGTGCGCCCCTC | |
| 52 | chr21.21755916.21755918 | GCCCCGCATCCAGGGCAGGGGGACTGCGACTGCCAG | 52 |
| | | AGGCTGAGCAACTCGCC | |
| 53 | chr12.1420136.1420138 | CCAGTCTGAAACGGCTGCTAGGAACCCGATCAAACCC | 53 |
| | | TTCACAAATTTTCTCA | |
| 54 | chr9.49065853.49065855 | ACCCTCGGGGGCCCTACCGGCAGCGCCGCCGGAGCTT | 54 |
| | | GAGCCGCGCCGACCGT | |
| 55 | chr9.5243304.5243306 | GCTCCAGCGCCCCGGGCCTGTCTGGGCGGCCGGGAGC | 55 |
| | | GCCCCGCCTGGGACCA | |
| 56 | chr33.16612511.16612513 | CGGGCTCGGCACTTGGCAGAGGTTCGCGTTCCCAGGT | 56 |
| | | GCCCTGGGGCCCCCGC | |
| 57 | chr19.30633446.30633448 | GCTGAAGCCTATGGTCTTCCTGCCTGCGACACGCGTT | 57 |
| | | TCATTGGCAAGCAAGT | |
| 58 | chr4.24322396.24322398 | CCGGCCCCCGCCCTCACACTGACTTTCGGAAGATACA | 58 |
| | | GGGGAAGAAGGGGTCC | |
| 59 | chr1.112644846.112644848 | CCTCGGGGCTCAGGCCAAGCCCACAGCGCGCGGGGG | 59 |
| | | AGGGATGCTCGGGCAGG | |
| 60 | chr25.36026237.36026239 | GCAGGGGAATTGGACCCGCGGGGACTCGCGCCTTCCC | 60 |
| | | GGACCGGCGGCGGGGA | |
| 61 | chr9.6335033.6335035 | GTGCCTAGGTAACAGTCCACCTAACGCGAGGTGCAG | 61 |
| | | GCCAGCTCTCCCAAGAT | |
| 62 | chr25.47771926.47771928 | AAGCTCCCCCGTCTCCCGCTGACGGGCGGCCGGCTGC | 62 |
| | | AGGCGTCGGGACGCCA | |
| 63 | chr28.15361862.15361864 | AAATTGGGACTTGAACCCAGTTCTGACGCTGGAGAAA | 63 |
| | | GCGATTTTAACACTAT | |
| 64 | chr10.19454640.19454642 | CGTGTGCAGCCGCGCCCAGCACGGGCCGCCCCTCCCG | 64 |
| | | GACCTGAGCTCGCGCC | |
| 65 | chr24.45976254.45976256 | GGGCGCCGCGCCCGCGGGGAGCTCGCCGCCCGGGCC | 65 |
| | | GCCCTCGGGCCCCCGCG | |
| 66 | chr20.8376423.8376425 | GCTGGTCCAGGGTGGGCTGCAGGGCCCGGGACACTT | 66 |
| | | GCTTGGCCCTCTCCCCG | |
| 67 | chr22.9461909.9461911 | GGCACGCACCCCCGGAGGCGAGGTGTCGGCCAGAGG | 67 |
| | | GGGGACCGTCAGGGGGT | |
| 68 | chr6.16959645.16959647 | CGCCCCCTTCCCAGCCTCAGCGGCCTCGCCTGGGCCT | 68 |
| | | CCGCCAGCGTGAGCAC | |
| 69 | chr5.32339666.32339668 | GGGCCGCAGGACTGCTCGAAAGGCATCGGATTACCC | 69 |
| | | AGAATAACTCCCAAATA | |
| 70 | chr5.58042813.58042815 | CGGCAGGGCCGGAGCGGGGGACTGGCCGGCTGGACC | 70 |
| | | CAGGGCGCCGACCCACC | |
| 71 | chr5.69901226.69901228 | ACCTGCTGCGCATGAAGGTGCGGGGACGGCGGGGAG | 71 |
| | | GCGCGCTGGGCGACGGG | |
| 72 | chr15.2515117.2515119 | GGCCGTGGGGCTCAGGTGTGAGCGGCCGCCGAGGCG | 72 |
| | | CGCGCCCCGAGTGTGCG | |
| 73 | chr7.66213015.66213017 | GCCAGTGTTCCCAAGAGATGCTCAGCCGCTAGTGACA | 73 |
| | | GGTTTAGCTCCACTTT | |
| 74 | chr23.12007637.12007639 | GGCCCTGCTGCCCGGCCCTGGCCGCCCGGCCTCAGAT | 74 |
| | | CTTCCCCCGGCGGGCG | |
| 75 | chr8.14950723.14950725 | TCCGCTCCCTCTCCCGACCCCCGCCGCGGTCTTCCGCT | 75 |
| | | TACTCACAGGGGTAG | |
| 76 | chr10.17323714.17323716 | GCCGCGCTGGCGGGACCCCGTGACTGCGGACCGCGG | 76 |
| | | GCCCTCTCCGATGCACT | |
| 77 | chr11.12032331.12032333 | GCTTGACCAGGGGCCGCTGAGCAGGACGTGCTCTGCA | 77 |
| | | GGCACACTAAGCTCAC | |
| 78 | chr30.19450208.19450210 | AGAGACAGTGATGAGGAATGAGCTGCCGAAGGAATT | 78 |
| | | TGGCAAGGAGGGGAGCG | |
| 79 | chr9.37505164.37505166 | TATCTCTGCGTGCTTTGCATCCTAGTCGAATTGCTTAG | 79 |
| | | TTGGATGAAATGTCA | |
| 80 | chr10.17323692.17323694 | CCGCGCTCGGGCGAGGGGCCAGGCCGCGCTGGCGGG | 80 |
| | | ACCCCGTGACTGCGGAC | |
| 81 | chr4.25745353.25745355 | GCGCTCCGTGCTGGGTGCTCCGCGCTCGGTGCTCCGC | 81 |
| | | GCCCCCACTCGGTGCT | |
| 82 | chr5.56402692.56402694 | CAGCTCCCTCGAGAGAGTGTTCTCTGCGGTGACCAGG | 82 |
| | | AAGGTGGGAAGGTGCT | |
| 83 | chr13.38036362.38036364 | CAACGGGGTCGCAGCAGCGCACAGTGCGCGGGAACC | 83 |
| | | CTGCTTTCCCGAGGCAG | |
| 84 | chr12.8645238.8645240 | GGAGAGCGACTGCGCACGCGCCGGGTCGAGTTCTGG | 84 |
| | | GCACCACGTTTAGCTCC | |
| 85 | chr13.37468055.37468057 | TCGGGCATCTCAGGCATCCGAGGGGGCGGCCTGGCTG | 85 |
| | | TGCGCAGAGGAGCCCA | |
| 86 | chr9.37133899.37133901 | GTGGGAGCAGGAGGTCAGCGCAGGCCCGTCGCCCCC | 86 |
| | | GGGGAAAAGCGGGGTAC | |
| 87 | chr28.40315231.40315233 | GGCCGAGCCCCCTCCCCACGCTGCCCCGCTGGACACG | 87 |
| | | AGGGCGCAGGCGGGAG | |
| 88 | chr5.3784935.3784937 | AGAATTCTTCTATGACTCGTCTTTTTCGTTCCCTTCGT | 88 |
| | | TTATTTCCCGAAGTC | |
| 89 | chr20.48020143.48020145 | GTCAGCTCTTCCGGACGTGGCGAGTTCGCCTCTGCCC | 89 |
| | | GAAACTCATCCCGCGT | |
| 90 | chr6.20969749.20969751 | GCATGGAGCACATCACCTAAGTAAGCCGGCCTTCCGT | 90 |
| | | TGGCGGACAGCCCGAG | |

1: A method of determining whether a canine subject has or is at a risk of developing osteoarthritis (OA), the method comprising:
a) providing a DNA methylation profile from a sample obtained from the canine subject; and
b) determining whether the canine subject has or is at risk of developing OA by comparing at least part of the DNA methylation profile to a reference DNA methylation profile.

2: The method according to claim 1, wherein step a) comprises determining a DNA methylation profile from a sample obtained from the canine subject.

3: The method according to claim 2, wherein DNA methylation is determined according to a method which comprises: (i) one or more of the following steps: (a) treating the sample DNA with APOBEC or using bisulfite conversion to deaminate unmethylated cytosines; (b) a capture-based enrichment; and/or (c) high throughput sequencing or arrays; or (ii) de novo sequencing.

4: The method according to claim 1, wherein in step a) the sample is a blood sample.

5: The method according to claim 1, wherein the DNA methylation profile comprises at least one methylation site as listed in Table 1.

6: The method according to claim 1, wherein the DNA methylation profile comprises at least 2, at least 5, at least 10, at least 25, at least 50, at least 60, at least 70, at least 80 or each of methylation sites as listed in Table 1.

7: The method according to claim 1, wherein the DNA methylation profile comprises methylation sites listed as site numbers 1 to 5 in Table 1.

8: The method according to claim 1, wherein the DNA methylation profile comprises methylation sites listed as site numbers 1 to 10 in Table 1.

9: The method according to claim 1, wherein the DNA methylation profile comprises methylation sites listed as site numbers 1 to 25 in Table 1.

10: The method according to claim 1, wherein the DNA methylation profile comprises methylation sites listed as site numbers 1 to 50 in Table 1.

11: The method according to claim 1, wherein the DNA methylation profile comprises methylation sites listed as site numbers 1 to 60 in Table 1.

12: The method according to claim 1, wherein the DNA methylation profile comprises methylation sites listed as site numbers 1 to 70 in Table 1.

13: The method according to claim 1, wherein the DNA methylation profile comprises methylation sites listed as site numbers 1 to 80 in Table 1.

14: The method according to claim 1, wherein the DNA methylation profile comprises each of methylation sites listed in Table 1.

15: The method according to claim 1, wherein the DNA methylation profile of the canine subject is compared to a reference DNA methylation profile using machine learning.

16: The method according to claim 1, wherein the DNA methylation profile comprises at least one OA specific DNA methylation marker.

17: A method for selecting a dietary, pharmacological, or lifestyle regime for a canine subject, the method comprising:
a) determining whether the canine subject has or is at risk of developing osteoarthritis (OA) by the method of claim 1; and
b) selecting a suitable dietary, pharmacological, or lifestyle regime for the canine subject based on the whether the canine subject has, or is at risk of developing, OA as determined in step a).

18: The method according to claim 17, wherein the method further comprises administering the selected dietary, pharmacological, or lifestyle regime to the canine subject.

19: A method for preventing or reducing the risk of a canine subject developing osteoarthritis (OA); the method comprising:
a) determining whether the canine subject has or is at risk of developing osteoarthritis (OA) by the method of claim 1; and
b) selecting a suitable dietary, pharmacological, or lifestyle regime for the canine subject based on the whether the canine subject has, or is at risk of developing, OA as determined in step a);
wherein the dietary, pharmacological, or lifestyle regime prevents or reduces the risk of the canine subject developing OA.

20: The method according to claim 19, wherein the method further comprises administering the selected dietary, pharmacological, or lifestyle regime to the canine subject.
